(19) United States
(12) Patent Application Publication
(10) Pub. No.: US 2009/0130033 A1
(43) Pub. Date: May 21, 2009

(54) Dendrimer-Aminobuadiene-Based Uv Screens

(76) Inventors: Sebastianus Gerardus Kluijtmans, Zeist (NL); Jan Bastiaan Bouwstra, Bilthoven (NL)

Correspondence Address: FOLEY AND LARDNER LLP;SUITE 500, 3000 K STREET NW, WASHINGTON, DC, 20007, US

(21) Appl. No.: 11/572,560

(22) PCT Filed: Jul. 25, 2005
(86) PCT No.: PCT/NL05/00538

§ 371 Date: Jan. 23, 2008

(30) Foreign Application Priority Data
Jul. 23, 2004 (EP) 04077128.9

(51) Int. Cl. A61K 8/81; C08F 136/00; A61Q 17/04

(52) U.S. Cl. 424/59; 526/310; 977/754

(57) ABSTRACT

The invention relates to UV-absorbing polymers, said UV-absorbing polymer comprising a synthetic amine rich polymer to which is covalently linked to an aminobutadiene represented by the general formula (I) wherein the UV-absorbing polymer has a number average molecular weight Mₙ of 1000 g/mol to 100.000 g/mol; and said UV-absorbing polymer having a UV-absorption of at least 5.6 a.u./g.L at 375 nm.

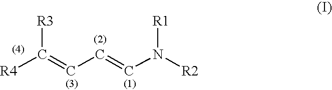

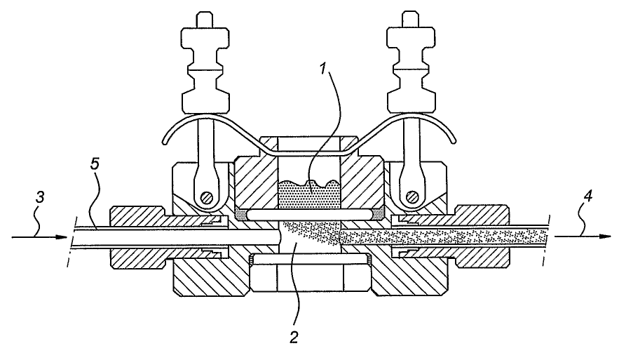

### FIELD OF THE INVENTION

The present invention is in the field UV-absorbing polymers, in particular useful in sunscreen compositions.

### BACKGROUND OF THE INVENTION

The detrimental effects of exposing the skin to UV light are manifold and are well documented in the prior art. It has been long recognized that UV-B radiation, with a wavelength of 290 to 315 nm, causes erythematic disorders or sunburn. It was not until around 1980 that it was discovered that UV-A radiation, with a wavelength from 315 to 400 nm, causes phototoxic and photochemical reactions.

While about 70% UV-B radiation is blocked by the outer skin or stratum corneum, this is not the case for UV-A radiation, which can subsequently penetrate deep into the living dermis. A well known destructive effect of UV-A is oxidative stress. Superoxide, which is formed by UV-A radiation, can release iron from ferritin, an iron-storage protein located in fibroblasts in the skin (Pourzand et al, Proc. Natl. Acad. Sci. USA, June 1999, Vol 96, p. 6751-56). The role of iron in the Fenton or Haber Weis reaction resulting in the production of highly destructive hydroxyl radicals and hydrogen peroxide is well known. Also other metal ions like copper-ions have been reported to catalyze the formation of oxygen radicals. The role of these destructive products in damaging DNA is well known as for example described by Sestili et al (Free Radical Biology & Medicine [US], Jul. 15, 1998, 25, [2] p. 196-200).

The normal biochemical protection by enzymes like superoxide dismutase is not sufficient to effectively stop the reaction induced by UV-A radiation. Hence the necessity to protect the skin from these harmful effects is still mandatory. Other oxidative stress phenomena are damaging of collagen resulting for example in accelerated skin aging and white spots, or damaging of cell walls by lipid peroxidation.

The use of organic UV-absorbers for sunscreen applications is widely known. A disadvantage of organic UV-absorbing compounds is their low water solubility. Lipid soluble UV-absorbing compounds, with or without the combination with organic solvents are capable of passing the so-called stratum corneum with the risk of entering the bloodstream.

A further disadvantage of UV-absorbing compounds is that they are unstable under UV-light. UV-exposure can cause photochemical reactions that destroy the UV-absorbing polymer thereby reducing the protection against UV radiation.

U.S. Pat. No. 4,839,160 discloses a cosmetic formulation for protecting the skin against UV-radiation comprising a polymer of benzylidenbornanone units having C₄-C₁₂ alkoxy chains. This approach does not result in an increase of the hydrophilicity of the complex. The effect on the penetration through the skin is disclosed, however, the increase of the hydrophobicity will increase the mobility through the lipid elements in the stratum corneum instead of the other way around.

The coupling of UV-A and UV-B absorbing compounds to a polyacrylic acid through an oxygen or nitrogen atom, forming substantially insoluble particles is disclosed in WO 01/08647, resulting in a molecular complex designed specifically for the uptake of a-polar compounds to adapt the refractive index into a desired direction. However, the complex has a limited UV absorption and mixing with oily compounds has the risk of skin penetration in case of smaller MW complexes that could result in undesired immunogenic reactions.

WO 02/92668 discloses hyperbranched or dendrimeric polymers to which functional groups can be coupled as plastic or coating additives, but is silent with respect to cosmetic applications.

WO 00/65142 discloses polymers with broad UV absorption for protection of textile or skin by adhering the polymers to textile fibres, but is silent with respect to application directly on the skin, and mentions dendrimers only as a possible spatial distribution of the UV-absorbing monomers.

EP A 1.172.399 discloses a method to couple organic compounds to a polymer which has at least one reactive amine group. In this document the use of the synthesized materials for sunscreen applications is not mentioned.

FR A 2.757.389 discloses the combined use of UV-absorbers and a hyperbranched or dendrimeric polymer in sunscreen formulations.

WO 04/006878 discloses merocyanine derivatives for cosmetic use, in particular sunscreen formulations.

WO 04/075871 discloses cosmetic compositions for protection against UV radiation comprising for example a product obtained by coupling aminobutadienes and a polymer having free amino groups.

In spite of these attempts, there remains a need for UV filters which provide sufficient direct protection from sunlight by blocking the radiation while not showing any penetration through the skin and being fully transparent for the visible light radiation.

### SUMMARY OF THE INVENTION

It is an object of this invention to provide a new, highly efficient, UV-absorbing polymer.

It is a further object of the invention to provide a UV-absorbing polymer that can easily be formulated into a sunscreen composition without giving viscosity and/or stability problems.

A further object of the invention is to provide a sunscreen composition comprising a UV-absorbing polymer, which is substantially transparent in the visible light region.

Another object of the invention is to provide a sunscreen composition comprising a UV-absorbing polymer that can be applied in high concentrations and that are stable against breakdown under UV-light.

Another object of the invention is to provide a sunscreen composition comprising a UV-absorbing polymer which cannot penetrate through the skin, subsequently minimizing any risk of immunogenic or allergic side reactions.

A further object of the present invention is to provide a UV-absorbing polymer having a high solubility in cosmetic ingredients such as cosmetic oils and having a low skin penetration.

Surprisingly it was found that by coupling an aminobutadiene of general formula (I) to a suitable synthetic amine rich polymer a UV-absorbing polymer was obtained with an unexpected high and efficient UV-absorption. It was found that in order to function properly in sunscreen compositions this UV-absorbing polymer should have a UV-absorption of at least 5.6 a.u./g.L at 375 nm.

Thus the invention relates to a UV-absorbing polymer, said polymer comprising a synthetic amine rich polymer to which is covalently linked to an aminobutadiene represented by the general formula (I):

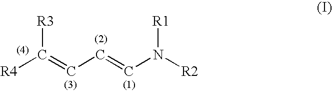

wherein R₁ and R₂, which may be the same or different, each represents a hydrogen atom, an alkyl group having 1-20 carbon atoms, an acyl group having 1-10 carbon atoms, or an aryl group having 6-20 carbon atoms, provided that R₁ and R₂ do not simultaneously represent hydrogen atoms, optionally R₁ and R₂ can combine and form a cyclic amino group or R₁ or R₂ can combine with C(1) and from a cyclic amino group;
each of R₁ and R₂ may be substituted by one or more carboxylic acid moieties;
R₃ represents —COOH, —COOR₅, —COR₅, —CN or SO₂R₅ and R₄ represents —COOH, —COOR₆, —COR₆, —CN or —SO₂R₆ wherein R₅ and R₆, which may be the same or different, each represents an alkyl group having 1-25 carbon atoms or an alkyl group having 1-25 carbon atoms and one or more carbon-carbon double bonds, wherein the alkyl group optionally comprises one or more hetero-atoms selected from the group consisting of O, N, Si and S, or an aryl group having 6-20 atoms; optionally R₅ and R₆ can combine and form a ring structure, said ring structure preferably being a five to seven membered ring structure, which is optionally substituted and which optionally comprises N, O and/or carbonyl groups,
and said polymer having a UV-absorption of at least 5.6 a.u./g.L at 375 nm.

Further, the invention relates to a method for the preparation of a UV-absorbing polymer according to the invention, said method comprising contacting an aminobutadiene of general formula (I) with a carboxylic acid and coupling the resulting product with a synthetic amine rich polymer.

The invention further relates to a method for the preparation a UV-absorbing polymer according to the invention, said method comprising coupling a derivative of an aminobutadiene of general formula (I) with a synthetic amine rich polymer.

Also the invention relates to the use of a UV-absorbing polymer for the preparation of a cosmetic composition for protection against UV radiation.

### DETAILED DESCRIPTION OF THE INVENTION

The present invention relates to UV-radiation absorbing polymers that can be used in compositions to protect the human skin or hair from the detrimental effects of exposure to sunlight. For the UV-absorbing polymer to be useful in sunscreen formulations that have sufficient protection against UV-radiation, the UV-absorption of the polymer should be at least 5.6 a.u/g.L at 375 nm. A lower absorption would require a higher amount of the UV-absorbing polymer in the sunscreen composition giving rise to formulation problems such as stability and viscosity problems.

By coupling an aminobutadiene according to the general formula (I) to a suitable synthetic amine rich polymer to provide a UV-absorbing polymer it was found that the high absorption of the aminobutadiene according to the general formula (I) was retained and that the synthetic amine rich polymer could be loaded with a sufficient amount to achieve an UV-absorption of the UV-absorbing polymer of at least 5.6 a.u/g.L at 375 nm. This minimum UV-absorption is required to avoid any detrimental effects on the stability and viscosity of the sunscreen formulation.

### The Aminobutadiene According to General Formula (I)

The aminobutadiene according to the present invention has the general formula (I) disclosed above. A preferred class of aminobutadienes is that in which R₃ represents —COOH, —COOR₅, —COR₅ or SO₂R₅ and R₄ represents a carboxyl group, —COOR₆, —COR₆ or —SO₂R₆, wherein R₁, R₂, R₅ and R₆ have the meanings indicated above. Examples of aminobutadienes according to the general formula (I) are for example disclosed in U.S. Pat. No. 4,195,999. Table 1 lists a number of aminobutadienes according to the present invention which can be coupled to an amine rich polymer, including their absorption maxima in methanol (or ethanol when not soluble in methanol).

TABLE 1

| | General structure: R₁ and R₂ do not form a ring 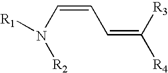 | Absorption maximum (nm) |
|---|---|---|
| 1.1i | 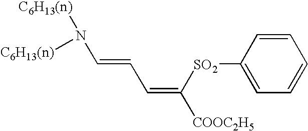 | 373 |
| 1.2i | 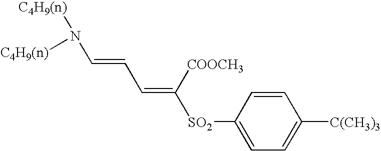 | 373 |
| 1.3i | 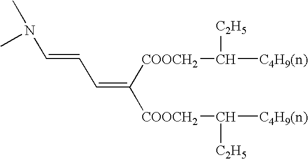 | 374 |
| 1.4i | 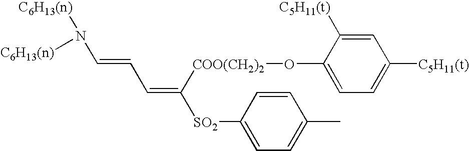 | 374 |
| 1.5i | 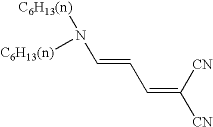 | 378 |
| 1.6i | 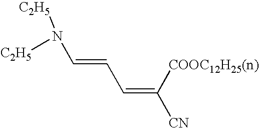 | 380 |
| 1.7r | 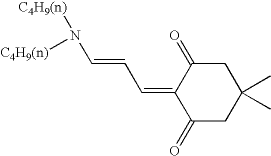 | 389 |
| 1.8r | 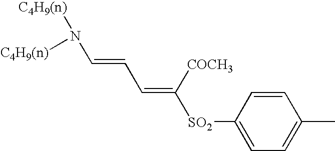 | 385 |
| 1.9r | 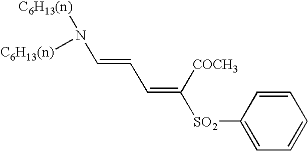 | 385 |
| 1.10r | 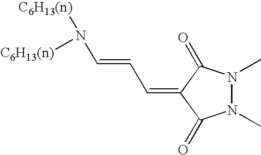 | 387 |
| 1.11r | 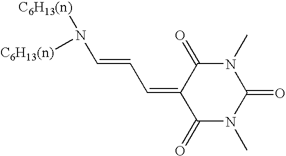 | 388 |
| 1.12r | 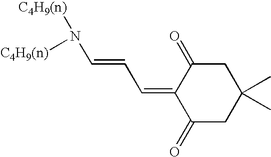 | 389 |
| 1.13r | 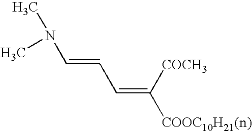 | 394 |
| 1.14r | 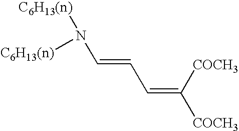 | 395 |
| | General structure: R₁ and R₂ together form a ring 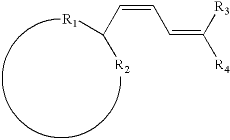 | Absorption maximum (nm) |
| 2.1 | 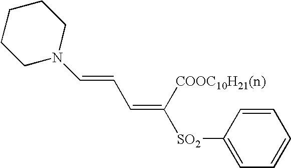 | 375 |
| 2.2 | 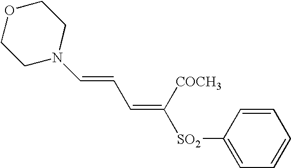 | 383 |
| 2.3 | 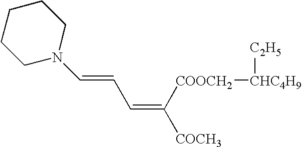 | 395 |
| | General structure: R₁ (or R₂) forms a ring with C(1) | |
| | 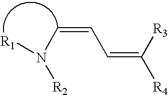 | |
| 3.1 | 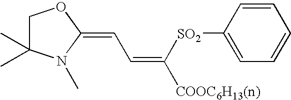 | 362 |
| 3.2 | 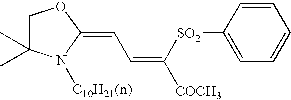 | 373 |
| 3.3 | 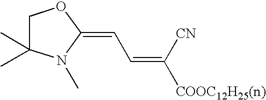 | 374 |
| 3.4 | 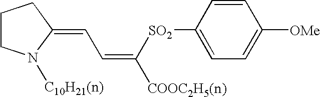 | 375 |
| 3.5 | 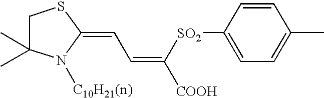 | 384 |
| 3.6 | 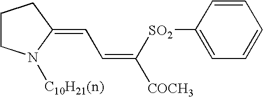 | 392 |
| 3.7 | 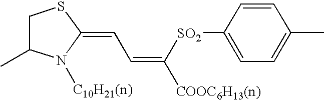 | 392 |

Of the aminobutadienes according to the general formula (I), an even more preferred class is that represented by the following general formula (II):

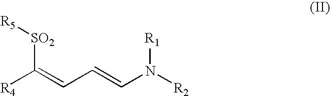

wherein R₁, R₂, R₄ and R₅ have the same meaning as in the general formula (I) It was found that the presence of an —SO₂R₅ group improves the stability of the compounds under UV-light.

A yet even more preferred class of aminobutadienes according to formula (II) is that wherein R₄ is —COOR₆, wherein R₆ has the meaning identified above.

A most preferred class of aminobutadienes according to the present invention is represented by the general formula (III), wherein R₁, R₂ and R₅ are as defined above and wherein R₆ is a linear or branched, preferably linear, alkyl group having 10-20 carbon atoms, even more preferably 12-20 carbon atoms en most preferably 12-18 carbon atoms:

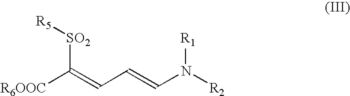

An example of the aminobutadiene according to the general formulas (I) and (II) is:

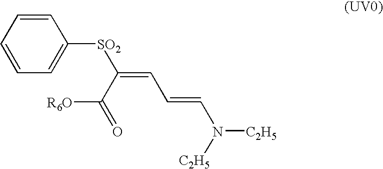

wherein R₆ has the same meaning as in general formula (I). Hereinafter the compound UV0 wherein R₆ is ethyl is referred to as UV1.

Another example of the aminobutadienes according to the invention has the following structure formula:

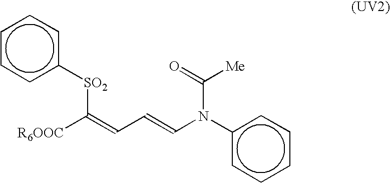

wherein R₆ has the same meaning as in the general formula (I). For example, in the compound UV2 wherein the group R₆ is ethyl is referred to as UV3. Compounds like UV2 and UV3 are very suitable for coupling the aminobutadienes according to the present invention to the synthetic amine rich polymers as will be elaborated below.

### The Synthetic Amine Rich Polymer

The synthetic amine rich polymer according to the present invention may be any amine rich polymer that is capable of coupling a sufficient amount of aminobutadiene to result in a UV-absorbing polymer having an UV-absorption of at least 5.6 a.u/g.L. Preferably, amine rich polymers that are suitable for cosmetic compositions, in particular sunscreen compositions are used. Such polymers are known to the skilled person and comprise hyperbranched and dendrimeric polymers. Preferably, the hyperbranched polymers and dendrimeric polymers have terminal primary or secondary, preferably primary, amino groups Hyperbranched polymers are obtained from the random polymerization of monomers in the presence of at least one polyfunctional monomer capable of introducing branching. Such a synthetic scheme is shown by Hawker and Devonport in “Step-Growth Polymers for High-Performance Materials: New Synthetic Methods,” Hedrick, J. L. and Labadie, J. W., Eds., Am. Chem. Soc., Washington, D.C., 1996, pp. 191-193. Hult et al., in “Advances in Polymer Science,” Vol. 143 (1999), Roovers, J., Ed., Springer, New York, pp. 1-34, present a review of hyperbranched polymers.

Highly branched dendritic polymers are well known, as discussed for example in “Polymeric Materials Encyclopedia”, Vol. 5 (1996), J. C. Salamone, Ed., CRC Press, New York, pp. 3049-3053. Dendritic polymers have a non-linear architecture and are intrinsically globular in shape. Discrete, stepwise synthetic methods are used to prepare highly branched pure compounds or dendrimers. As discussed by Hawker and Devonport in “Step-Growth Polymers for High-Performance Materials: New Synthetic Methods”, Hedrick, J. L. and Labadie, J. W., Eds., Am. Chem. Soc., Washington, D.C., 1996, pp. 186-196, if the macromolecule has highly regular branching which follows a strict geometric pattern, it is a dendrimer. Dendrimers are typically monodisperse and are prepared in a multi-step approach with purifications at each stage. The architecture of dendrimers is also discussed by Roovers and Comanita in “Advances in Polymer Science”, Vol. 142 (1999), Roovers, J., Ed., Springer, New York, pp. 179-228. Dendrimers consist of a core molecule which defines the centre of symmetry of the molecule, and branching layers. Tomalia et al., Angew. Chem. Int. Ed. Eng., 29 (1990), 138-175 disclose “starburst” dendrimers which consist of an initiator core and branching groups.

Dendrimers, which are globular, are preferred because their solution viscosity increases less with concentration than linear polymers. Hyperbranched polymers are irregular and have globular as well as linear characteristics.

In one embodiment the synthetic amine rich polymer is a dendrimer having free amine groups to couple to the aminobutadiene according to the general formula (I). The dendrimer can be produced by divergent growth or convergent growth or a combination of these. To obtain a high enough molecular weight for preventing skin penetration, at least 1^{st} generation dendrimers are preferably used. More preferably, at least 2^{nd} generation and yet more preferably not higher than 4^{th} generation dendrimers are used. It is, however, also possible to start with a core molecule used to built a dendrimer, but now use this core for the addition UV absorbers having aliphatic chains and by this achieving a high molecular weight. Cost and complexity of synthesis will increase with higher generations. A too high molecular weight is less preferred due to the effect on properties like for example solubility and viscosity. The degree of branching of the dendrite depends on the number of functional groups present on the monomers. Preferably, monomers designated AB₂, which result in two branches per addition, are used. More preferably monomers AB₃ are used, resulting in three branches per addition. The core molecule, or generation 0 of the dendrimer, can have two functional groups to which monomers ABₓ are added, but preferably has at least three functional groups, even more preferably at least four.

Suitable amine rich polymers, in particular dendrimers having terminal amine functional groups, are disclosed in U.S. Pat. No. 4,694,064, U.S. Pat. No. 4,507,466, U.S. Pat. No. 4,631,337, U.S. Pat. No. 4,558,120, U.S. Pat. No. 4,568,737, U.S. Pat. No. 4,587,329; WO 95/02008, WO 93/14147, EP A 234.408, U.S. Pat. No. 4,289,872, U.S. Pat. No. 4,360,646, Proc. Natl. Acad. Sci. USA, 85 5409-5413 (1988), which are all incorporated herein by reference.

Suitable commercially available dendrimers are for example Astramol™ from DSM and Starburst™ from Dow Chemical, Inc.

In addition to coupling of aminobutadienes to the exterior end groups of a dendrimer interior modification can be done. For example, hydroxyl groups can be introduced to increase hydrophilicity of the dendrimer-aminobutadiene complex.

In another embodiment the synthetic amine rich polymer is a hyperbranched polymer having free amine groups to couple to the aminobutadiene according to general formula (I).

According to the present invention, the synthetic amine rich polymer is most preferably represented by the general formulas (IV)-(VII):

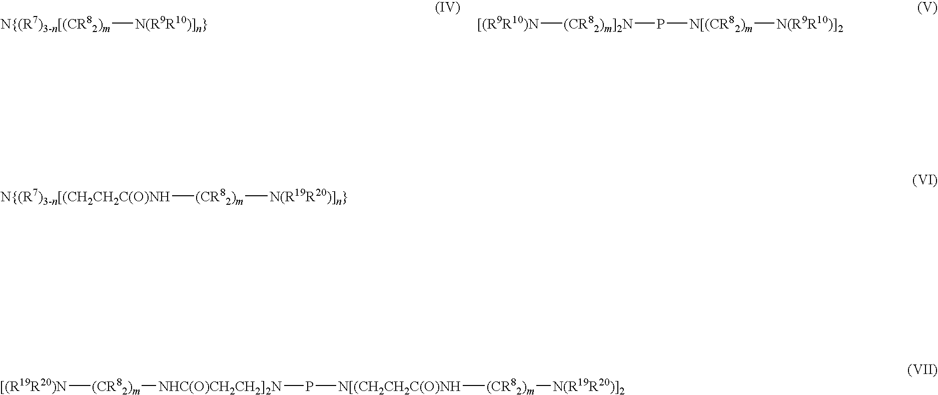

wherein R⁷ is a hydrogen atom, a linear or branched C₁-C₂₀ alkyl group or a —[(CR¹⁷₂)_{q}—X]ₒ—R¹⁸ group wherein X is O or NH;
m is 2, 3 or 4;
n is 2 or 3;
o is 1-10;
q is 2 or 3;
P is —(CR⁸₂)ₘ—, a C₆-C₁₂ arylene group, a C₆-C₁₂ cycloalkylene group or a —[(CR¹⁷₂)_{q}—X]ₚ—C(R¹⁷)₂]— group wherein X is O or NH and p is 1-10;
R⁸ is a hydrogen atom or a linear or branched C₁-C₆ alkyl group;
R⁹ and R¹⁰ are independently a hydrogen atom, a linear or branched C₁-C₆ alkyl group or a group of the formula (CR¹⁷₂)_{q}NR¹¹R¹², provided that R⁹ and R¹⁰ are not both a linear or branched C₁-C₆ alkyl group;
R¹¹ and R¹² are independently a hydrogen atom, a linear or branched C₁-C₆ alkyl group or a group of the formula (CR¹⁷₂)_{q}NR¹³R¹⁴, provided that R¹¹ and R¹² are not both a linear or branched C₁-C₆ alkyl group;
R¹³ and R¹⁴ are independently a hydrogen atom, a linear or branched C₁-C₆ alkyl group or a group of the formula (CR¹⁷₂)_{q}NR¹⁵R¹⁶, provided that R¹³ and R¹⁴ are not both a linear or branched C₁-C₆ alkyl group;
R¹⁵ and R¹⁶ are independently a hydrogen atom or a linear or branched C₁-C₆ alkyl group, provided that R¹⁵ and R¹⁶ are not both a linear or branched C₁-C₆ alkyl group;
R¹⁷ is a hydrogen atom or a methyl group, provided that at least one R¹⁷ is a hydrogen atom;
R¹⁸ is a hydrogen or linear or branched C₁-C₂₀ alkyl group or a —[(CR¹⁷₂)_{q}—X]ₒ—R¹⁸ group as defined above;
R¹⁹ and R²⁰ are independently a hydrogen atom, a linear or branched C₁-C₆ alkyl group or a group of the formula —CH₂CH₂C(O)NH—(CR⁸₂)ₘ—N(R²¹R²²), provided that R¹⁹ and R²⁰ are not both a linear or branched C₁-C₆ alkyl group;
R²¹ and R²² are independently a hydrogen atom, a linear or branched C₁-C₆ alkyl group or a group of the formula —CH₂CH₂C(O)NH—(CR⁸₂)ₘ—N(R²³R²⁴), provided that R²¹ and R²² are not both a linear or branched C₁-C₆ alkyl group;
R²³ and R²⁴ are independently a hydrogen atom, a linear or branched C₁-C₆ alkyl group or a group of the formula —CH₂CH₂C(O)NH—(CR⁸₂)ₘ—N(R²⁵R²⁶), provided that R²³ and R²⁴ are not both a linear or branched C₁-C₆ alkyl group;
R²⁵ and R²⁶ are independently a hydrogen atom or a linear or branched C₁-C₆ alkyl group, provided that R²⁵ and R²⁶ are not both a linear or branched C₁-C₆ alkyl group.

In general, it is preferred that q is 2.

A preferred class of synthetic amine rich polymers according to formula (IV) is that wherein R⁸ is a hydrogen atom, n is 3 and m is 2 and wherein R⁹ and R¹⁰ are as defined above.

A more preferred class of synthetic amine rich polymers according to formula (IV) is that wherein R⁸ is a hydrogen atom, n is 3 and m is 2 and wherein R⁹ and R¹⁰ are independently a hydrogen atom, a linear or branched C₁-C₆ alkyl group or a group of the formula —(CR¹⁷₂)_{q}NR¹¹R¹², provided that R⁹ and R¹⁰ are not both a linear or branched C₁-C₆ alkyl group; and R¹¹ and R¹² are independently a hydrogen atom or a linear or branched C₁-C₆ alkyl group, provided that R¹¹ and R¹² are not both a linear or branched C₁-C₆ alkyl group.

A preferred class of synthetic amine rich polymers according to formula (V) is that wherein R⁸ is a hydrogen atom, m is 2 and wherein R⁹ and R¹⁰ are as defined above.

A more preferred class of synthetic amine rich polymers according to formula (V) is that wherein R⁸ is a hydrogen atom, m is 2 and wherein R⁹ and R¹⁰ are independently a hydrogen atom, a linear or branched C₁-C₆ alkyl group or a group of the formula —(CR¹⁷₂)_{q}NR¹¹R¹², provided that R⁹ and R¹⁰ are not both a linear or branched C₁-C₆ alkyl group; and R¹¹ and R¹² are independently a hydrogen atom or a linear or branched C₁-C₆ alkyl group, provided that R¹¹ and R¹² are not both a linear or branched C₁-C₆ alkyl group.

A preferred class of synthetic amine rich polymers according to formula (VI) is that wherein R⁸ is a hydrogen atom, m is 2, n is 3 and wherein R¹⁹ and R²⁰ are as defined above.

A more preferred class of synthetic amine rich polymers according to formula (VI) is that wherein R⁸ is a hydrogen atom, m is 2, n is 3 and wherein R¹⁹ and R²⁰ are independently a hydrogen atom, a linear or branched C₁-C₆ alkyl group or a group of the formula —CH₂CH₂C(O)NH—(CR⁸₂)ₘ—N(R²¹R²²), provided that R¹⁹ and R²⁰ are not both a linear or branched C₁-C₆ alkyl group; and R²¹ and R²² are independently a hydrogen atom or a linear or branched C₁-C₆ alkyl group, provided that R²¹ and R²² are not both a linear or branched C₁-C₆ alkyl group.

A preferred class of synthetic amine rich polymers according to formula (VI) is that wherein R⁸ is a hydrogen atom, m is 2 and wherein R¹⁹ and R²⁰ are as defined above.

A more preferred class of synthetic amine rich polymers according to formula (VI) is that wherein R⁸ is a hydrogen atom, m is 2 and wherein R¹⁹ and R²⁰ are independently a hydrogen atom, a linear or branched C₁-C₆ alkyl group or a group of the formula —CH₂CH₂C(O)NH—(CR⁸₂)ₘ—N(R²¹R²²), provided that R¹⁹ and R²⁰ are not both a linear or branched C₁-C₆ alkyl group; and R²¹ and R²² are independently a hydrogen atom or a linear or branched C₁-C₆ alkyl group, provided that R²¹ and R²² are not both a linear or branched C₁-C₆ alkyl group.

Most preferably, the synthetic amine rich polymer is represented by the general formulas (IV) and (V).

Since skin penetration should be limited as discussed above, the UV absorbing polymer should have a high molecular weight, which can be obtained by coupling many UV absorbing molecules to a low molecular weight dendrimer. Alternatively, the synthetic amine rich polymer it self may already have a high molecular weight, so that an UV absorbing polymer can be obtained by adding only a limited number of UV absorbing molecules to such synthetic amine rich polymers. The synthetic amine rich polymer has therefore a minimum number average molecular weight Mₙ of 1000 g/mol, or preferably 10.000 g/mol, and can even be as high as 80.000 g/mol. However, the maximum number average molecular weight Mₙ is 100.000.

### The UV-Absorbing Polymer

The UV-absorbing polymer according to the present invention is prepared by coupling the aminobutadienes to the synthetic amine rich polymers. Preferably the UV-absorbing polymer according to this invention has a UV-absorption of at least 5.6 a.u./g.L preferably 20 a.u./g.L, more preferably of al least 40 a.u./g.L, even more preferably of at least 60 a.u./g.L and most preferably of at least 80 a.u./g.L.

The aminobutadienes according to general formulas (I) and (II) may be coupled to a synthetic amine rich polymer, preferably via a carboxylic acid group in the aminobutadiene, using methods well known to one of ordinary skill. If the aminobutadiene does not contain a carboxylic acid group, such a group can be introduced by methods known by persons skilled in the art. Introducing the additional carboxylic acid group should obviously not affect the UV absorbing properties and is therefore preferably introduced at a position of the aminobutadiene that does not essentially interfere with the π-system of the aminobutadiene.

According to a first preferred process of the present invention, aminobutadiene according to general formula (I) is coupled via a modification of the amino group (NR₁R₂) which introduces an additional carboxylic acid functionality in this group. In particular the aminogroup is substituted by iso-nipecotic acid (4-piperidine carboxylic acid). It appears this substitution allows additional efficient coupling to the synthetic amine rich polymers and results in unexpected high absorption. Thus upon treatment of aminobutadiene according to general formula (I) with optionally, but preferably a base and contacting the resulting product with iso-nipecotic acid, an additional carboxylic acid functionality is introduced, which may be coupled via methods involving coupling via carboxylic acid functionalities known per se to a suitable synthetic amine rich polymer. A preferred method of coupling is via the activated ester, preferably the NHS ester, to amine functionalities on the amine rich polymer. Consequently, according to the first preferred process for the preparation of a UV-absorbing polymer, an aminobutadiene according to general formula (VII) is subjected to the following steps:
(a) reacting the aminobutadiene according to formula (VII), optionally but preferably in the presence of a base, with a carboxylic acid derivative according to formula (VIII) to form an aminobutadiene derivative according to formula (IX):

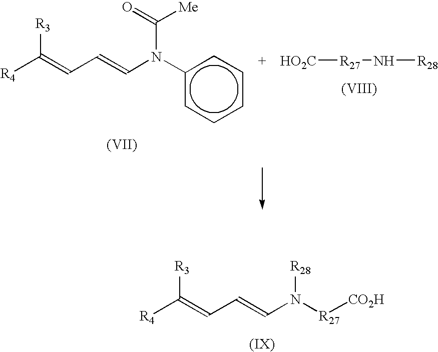

wherein R₂₇ and R₂₈, which may be the same or different, each represent an alkyl group having 1-10 carbon atoms; R₂₈ can also represent a hydrogen atom; optionally R₂₇ and R₂₈ can combine and form a cyclic amino group, preferably a five to seven membered amino group; and
(b) reacting the aminobutadiene derivative according to formula (IX) with a synthetic amine rich polymer according to any one of formulas (IV)-(VI) having —NHR* end groups to form a UV-absorbing polymer according to formula (X), wherein R* is hydrogen or a linear or branched C₁-C₆ alkyl group:

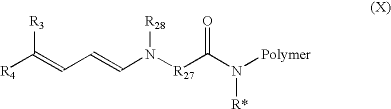

Suitable examples of the carboxylic acid derivative (VIII) are iso-nipecotic acid (4-piperidine carboxylic acid) and piperazine carboxylic acid.

According to a preferred embodiment of the first preferred method, the aminobutadiene derivative according to formula (IX) is first converted into an activated ester before it is reacted with the synthetic amine rich polymer. A preferred activated ester is the NHS-ester according to formula (XI):

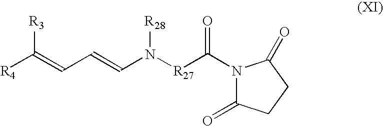

According to the invention, it is most preferred that the carboxylic acid derivative according to formula (VIII) is iso-nipecotic acid.

According to a second preferred method, the aminobutadiene according to general formula (I), preferably the aminobutadiene according to formula (VII), is coupled directly by mixing of the aminobutadiene with the amine rich polymer according to the invention having —NHR* end groups as defined above. However, this second preferred method proceeds with a somewhat lower efficiency as with the method comprising NHS activation of aminobutadiene.

It is not clear why aminobutadienes of formula (VII) couple spontaneously. It can be expected that similar structures having attached to the nitrogen group at least one substituted or non-substituted moiety with aromatic character and/or attached to the nitrogen at least one moiety with an electron-withdrawing character may couple spontaneously with adequate yield. Now that this simple method has been discovered, finding similar structures that can be coupled directly to the amine groups of polymers is a mere process of trial and error.

The present invention is also directed to the use of the UV-absorbing polymers according to the invention to protect the human skin or hair from the detrimental effects of exposure to sunlight. The present invention is especially directed to sunscreen compositions comprising stable UV absorbing polymers that cannot penetrate through the skin into the bloodstream. Preventing the UV absorbing polymers from entering the bloodstream reduces the risk of immune reactions or other detrimental effects.

In a preferred embodiment the UV-absorbing polymer absorbs less than 10% of its total absorption above 400 nm. This means that the UV-absorbing polymer is substantially transparent for visible light. In a preferred embodiment the UV-absorbing polymer has 75% or more of its total absorption in the UV-A region between 315 and 400 nm. Thus the invention relates to a UV-absorbing polymer as defined above wherein less than 10% of the total absorption between 250 and 600 nm of said UV-absorbing polymer is above 400 nm. Also the invention relates to a UV-absorbing polymer as defined above wherein at least 75% of the total absorption between 250 and 600 nm of said UV-absorbing polymer is between 315 and 400 nm.

In a preferred embodiment, the aminobutadiene according to the general formula (I) or formulas (VII), (IX) or (XI) is coupled to the synthetic amine rich polymer in an amount of at least about 40 mmol per 100 gram amine rich polymer, preferably in an amount of at least about 155 mmol per 100 gram amine rich polymer, more preferably at least about 350 mmol per 100 gram amine rich polymer, even more preferably at least about 605 mmol per 100 gram amine rich polymer and most preferably at least about 955 mmol per 100 gram amine rich polymer. The amount of aminobutadienes used can also be expressed as a percentage of amine groups in the synthetic amine rich polymer which is bonded to an amino butadiene. Most preferably, 100% of the amine groups is coupled to an aminobutadiene. Generally, good results can already be obtained when more than 50% of the amine groups is linked to an aminobutadiene. The coupled amounts necessary to reach the preferred UV-absorption may obviously vary between different aminobutadiene structures since said aminobutadiene structures can be expected to have varying extinction coefficients.

In another preferred embodiment, the coupling of the aminobutadiene according to the general formula (I) to a dendrimer or hyperbranched polymer is carried out with a method which does not show the risk of polymer cross-linking, increasing the MW of the UV-absorbing polymer in an uncontrolled manner. Especially the reaction of carbodiimide activator with a carboxygroup of the aminobutadiene according to the general formula (I) followed by conversion of the reactive intermediate to the N-hydroxysuccinimide ester which will react with an amino group in the amine rich polymer can result in a UV-absorbing polymer with the desired specifications. Such reactions are well known to the person skilled in the art (cf. for example J. March, Advanced Organic Chemistry, 4^{th} Ed., page 395, 1992).

The depth of penetration in the skin can be advantageously regulated by controlling the molecular weight of the dendrimer or hyperbranched polymer preventing it from entering the bloodstream. Preferably the UV absorbing polymer has a molecular weight of at least 1000 g/mol The number average molecular weight Mₙ may vary between wide limits and may be even as high as 100.000 g/mol. For practical reasons, e.g. the viscosity of formulations, a molecular weight between 1000 and 10.000 g/mol is preferred. Prior art applications of UV-absorbing polymers do not include a method to control the skin penetration, but allow skin penetration freely. In our invention even shallow penetration of the skin can be prevented.

Another method to regulate skin penetration is to adjust the hydrophobicity of the synthetic amine rich polymer by chemical modification of said polymer, covalently linking hydrophilic or hydrophobic groups to the polymer.

The UV-absorbing polymer can be advantageously used for the preparation of cosmetic or sunscreen compositions to protect the skin or hair from UV radiation.

Various forms of cosmetic compositions for skin protection comprising UV-absorbing polymers are available in the market as lotions, emulsions, creams, milks, gels and the like. These may contain oil and/or alcohol. Also aerosols or sticks are known to be used. All such forms of cosmetic compositions may function as a medium to apply the UV-absorbing polymer.

One skilled in the art will be able to select suitable cosmetic and dermatological acceptable carriers that can be used in the sunscreen composition of the invention.

The sunscreen composition comprising the UV absorbing polymer can contain a second, UV-absorbing group or compound as an additive or coupled to the synthetic amine rich polymer in addition to the coupled aminobutadiene. This second UV-absorbing group can be a UV-A, UV-B or broadband UV-absorbing compound as described in for example EP A 1.055.412. The second UV absorbing group can also be an aminobutadiene having complementary properties to the first coupled aminobutadiene. For example, a UV-A absorbing butadiene can be coupled and also a UV-B absorbing aminobutadiene. Other additives as applied in the art may also be used.

It will be clear to a person skilled in the art that the advantage of linking compounds to synthetic amine rich polymers like dendrimers or hyperbranched polymers is not limited to the aminobutadiene according to the general formula (I). For example, vitamins like vitamin C and vitamin E are normally added in cosmetic preparations as anti-oxidants. Although they do not impose a danger to the human organism, their function is reduced when such vitamins are allowed to diffuse away through the skin. Such compounds can also be advantageously linked to for example hyperbranched or dendrimeric polymers, optionally in combination with aminobutadienes.

The cosmetic compositions of the present invention can contain in addition to the UV-absorbing polymer various adjuvants conventionally present in cosmetic compositions of this type for example hydrating agents, emollients or thickening agents, surfactants, preservatives, perfumes, dyes, etcetera.

In one aspect the invention relates to a sunscreen composition comprising less than 18 wt. %, preferably less than 13 wt. %, more preferably less than 10 wt % of the UV absorbing polymer according to the invention, based on the total weight of the sunscreen composition. However, to be effective, the sunscreen composition must comprise at least 0.1 wt. % of the UV-absorbing polymer according to the invention, based on the total weight of the sunscreen composition.

### EXAMPLES

### Example 1

### Coupling of UV1 to Astramol™ (AM)32 Dendrimer Using Isonipecotic Acid

The NHS-ester of UV1 is prepared according to the following reaction

3.64 g UV1 and 1.18 g isonipecotic acid were dissolved in 20 ml dimethylsulfoxide (DMSO). The reaction mixture was heated for 3 hours at 80° C., and then cooled to room temperature. To this reaction mixture 1.05 g N-hydroxysuccinimide (NHS) and 1.88 g N,N-dicyclohexylcarbodiimide (DCC) were added. The mixture was stirred for 3 hours at room temperature. The formed dicyclohexylurea was filtered off. Thus the NHS-ester of UV1 was obtained in a DMSO solution.

### Coupling of UV1 to Astramol™ (AM)32 Dendrimer)

1.0 g Astramol™ (Am)32 was dissolved in 10 ml DMSO and added to the NHS-ester of UV1 in DMSO solution. After stirring overnight at room temperature the reaction mixture was added dropwise to 150 ml saturated solution of sodium carbonate in water. The solution was filtrated and the residue was washed with demineralized water. After drying in vacuo 5 g of the UV-absorbing polymer was obtained with a UV absorption of 110 absorption units/gram liter (A.U./gL) in DMSO, at a wavelength of 375 nanometer, measured on a HP 8452A diode array spectrophotometer with a sample concentration of 1 g/L and a light-pathway of 1 cm against a DMSO blanc.

### Example 2

### Coupling of UV3) Directly to Astramol™

To a solution of 1.0 g Astramol™ (Am)32 in 20 ml DMSO 3.58 g UV3 was added. After stirring overnight the reaction mixture was added dropwise to 150 ml saturated solution of sodium carbonate in water. The solution was filtered and the residue was washed with demineralized water. After drying in vacuo 4 g of the UV-absorbing polymer was obtained with a UV absorption of 130 absorption units/g.L (A.U./gL) in DMSO, at a wavelength of 375 nanometer, measured on a HP 8452A diode array spectrophotometer with a sample concentration of 1 g/l and a sample light-pathway of 1 cm. against a DMSO blanc.

### Example 3

### Coupling of UV1 to Linear Polyallylamine

The NHS-ester of UV1 in DMSO solution is prepared as in example 1.

2.6 g of a 20% solution of polyallylamine (Sigma Aldrich; 65,000 g/mol) was dissolved in DMSO and added to the NHS-ester of UV1 in DMSO solution. After stirring overnight at room temperature the reaction mixture was added dropwise to 150 ml saturated solution of sodium carbonate in water. The solution was filtered and the residue was washed with demineralized water. After drying in vacuo 5 g of the UV-absorbing polymer was obtained with a UV absorption of 108 absorption units/g.L (A.U./gL) in DMSO, at a wavelength of 375 nanometer, measured on a HP 8452A diode array spectrophotometer with a sample concentration of 1 g/l and a light-pathway of 1 cm. against a DMSO blanc.

### Example 4

### Penetration Depth of Aminobutadiene-Dendrimer into the Skin

A UV1-donor solution was prepared by dissolving 30 mg UV1 in 4 ml EtOH in 7 ml phosphate buffer (═PBS) pH 7.4.

Also donor solutions of UV(1) coupled to different generations dendrimers were prepared by dissolving 80 mg of UV1-dendrimer in 7 ml PBS buffer and 3 ml ethanol.

4 pieces of human skin (Ø ca 3 cm, mostly epidermis) were applied on a dialysis membrane and mounted into the diffusion cell shown in the figure. The figure is a schematic drawing of the diffusion cell, wherein 1=donor compound, 2=receptor compartment, 3=receptor input, 4=compound and receptor output for analysis, 5=⅛″ OD× 1/32″ wall tubing. The cells were mounted on a thermostatting and stirring device. The temperature was kept at 37°. The receptor fluid was a PBS buffer. The receptor compartment was stirred magnetically. 400 μl of the donor solution was applied. The receptor fluid flowing at a speed of with 1 ml/hr was collected into glass tubes (1 tube/2 hrs)

After 20 hrs the penetration experiment was stopped. UV-analysis of the collected receptor fluids indicated clearly the penetration of the UV-monomer UV1 through the skin while no penetration was measured for the UV-dendrimers (cf. Table 2).

TABLE 2

| | Presence in | |
|---|---|---|
| | receptor fluid | Presence in |
| | after 6 h | skin extract |
| 1. Aminobutadiene UV1 | + | + |
| 2. UV1-dendrimer based on Astramol | − | +/− |
| Am(8) UV-load 60 a.u./g.L | | |
| 3. UV1-dendrimer based on Astramol | − | − |
| Am(16) UV-load 60 a.u./g.L | | |
| 4. UV1-dendrimer based on Astramol | − | − |
| Am(32) UV-load 60 a.u./g.L | | |

Furthermore the skin parts were extracted with DMSO to dissolve any UV-absorber in the epidermis. The extracts were analysed by UV. The results, shown in table I second column, support the conclusion from the receptor fluids that the UV-dendrimers do not penetrate into or through the skin while the UV-monomer could be observed inside the skin sample.

In the above table a ‘+’ means that more than 50% of the substance penetrates the skin. A ‘−’ means that no detectable amount of the substance penetrated the skin. ‘±’ means that some of the substance was observed to penetrate into the skin, but did not enter the receptor fluid.

It is clear from these data that the aminobutadiene coupled to a dendrimer with a MW of more than 5 kD does not penetrate into the epidermis while the aminobutadiene coupled to a dendrimer with a molecular weight lower than 5 kD does penetrate to a limited extent. The uncoupled aminobutadiene showed the strongest penetration into the epidermis

### Example 5

### Stability of Aminobutadienes of General Formula (II)

The aminobutadiene molecules were applied in an acceptable sunscreen formulation and coated on a transparent support with a wet thickness of 24 μm. The concentrations of aminobutadiene molecules were chosen to reach an absorption at their absorption maximum (λmax) of 1.0 absorption units (A.U.) after drying (2 hrs at 40° C.). The samples were exposed to Xe-light (0.25 W/m2, Atlas) for 6 hrs after which the absorption was measured again, using a Hewlett Packard diode array spectrophotometer. Of each sample amount of absorption left after 6 hrs was compared to a control sunscreen formulation on a transparent support, that was not exposed to Xe-light. The results shown in Table 3 below, clearly indicate the beneficial effect of the presence of the SO₂ group on the light stability of the aminobutadiene UV-absorbers.

TABLE 3

| | Sunscreen formulation | Stability |
|---|---|---|
| UV-1 | 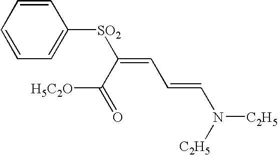 | + |
| 1.5i | 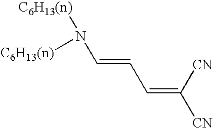 | − |
| 1.13r | 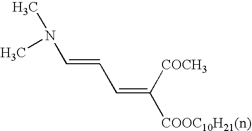 | − |
| 1.2i | 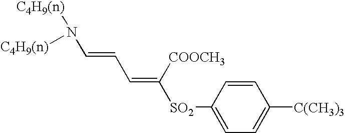 | + |
| 2.1i | 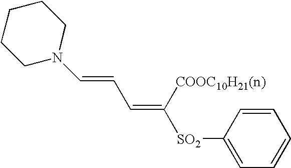 | + |

### Example 6

The following UV-absorbing polymers were tested on their solubility in a C₁₂-C₁₅ alkylbenzoate oil that is commonly used in sunscreen formulations at 40° C., by dissolving the UV polymers in the oil until saturation occurred and measuring the dissolved amount. A “+” indicates a solubility of more than 10 wt. %, a “−” indicates a solubility of less than 1 wt. %, and a “+/−” indicates a solubility of 1 to 10 wt. %. The data are summarised in Table 4.

TABLE 4

| UV-absorbing polymer | | |
|---|---|---|
| according to Structure (X) | | |
| wherein R₅ is phenyl and R* | | |
| is hydrogen: R₄ is as shown | Synthetic Amine | |
| below: | Rich Polymer | Solubility |
| C₂H₅ | (I) | − |
| n-C₈H₇ | (I) | − |
| n-C₁₆H₃₃ | (I) | + |
| n-C₁₈H₃₇ | (I) | + |
| n-C₁₈H₃₅ | (I) | + |
| C₂H₅ | (II) | − |
| n-C₁₆H₃₃ | (II) | + |
| n-C₁₀H₂₁ | (II) | +/− |
| n-C₈H₁₆ | (III) | − |
| n-C₁₆H₃₃ | (III) | + |
| C₂H₅ | (IV) | − |
| n-C₁₆H₃₃ | (IV) | + |

Structures Synthetic Amine Rich Polymers (Table 4):

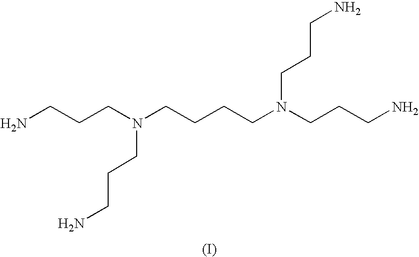

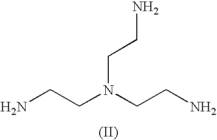

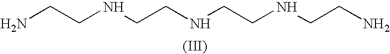

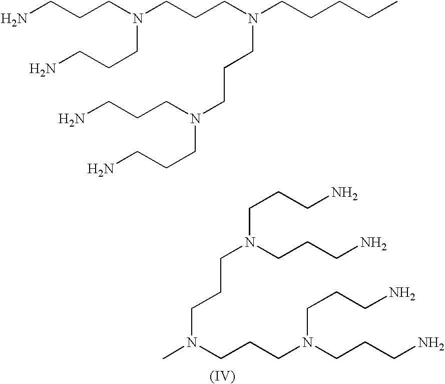

1-16. (canceled)

17. A UV-absorbing polymer comprising a synthetic amine rich polymer which is covalently linked to an aminobutadiene represented by formula (I): 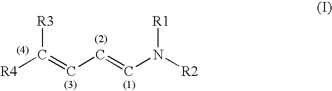
wherein R₁ and R₂, which may be the same or different, are each independently selected from the group consisting of hydrogen atoms, alkyl groups having 1-20 carbon atoms, acyl groups having 1-10 carbon atoms and aryl groups having 6-20 carbon atoms, provided that R₁ and R₂ do not simultaneously represent hydrogen atoms, optionally R₁ and R₂ can combine and form a cyclic amino group or R₁ or R₂ can combine with C(1) and from a cyclic amino group;
each of R₁ and R₂ are optionally substituted by one or more carboxylic acid moieties; R₃ is selected from the group consisting of —COOH, —COOR₅, —COR₅, —CN and SO₂R₅, and R₄ is selected from the group consisting of —COOH, —COOR₆, —COR₆, —CN and —SO₂R₆, wherein R₅ and R₆, which may be the same or different, are each independently selected from the group consisting of alkyl groups having 1-25 carbon atoms, alkyl groups having 1-25 carbon atoms and one or more carbon-carbon double bonds, wherein the alkyl groups optionally comprise one or more hetero-atoms selected from the group consisting of O, N, Si and S, and aryl groups having 6-20 atoms; optionally R₅ and R₆ can combine and form a ring structure which is optionally substituted and which optionally comprises N, O and/or carbonyl groups;
the UV absorbing polymer having a number average molecular weight Mₙ of 1,000 g/mol to 100,000 g/mol; and having a UV-absorption of at least 5.6 a.u./g.L at 375 nm.

18. The UV-absorbing polymer according to claim 17, wherein the aminobutadiene is represented by formula (II): 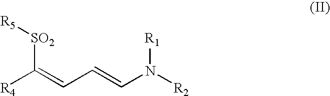
wherein R₁, R₂, R₄ and R₅ are as defined as in claim 17.

19. The UV-absorbing polymer according to claim 17, wherein the aminobutadiene is represented by the formula (III): 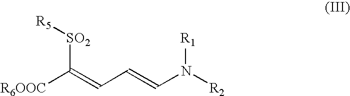
wherein R₁, R₂ and R₅ are as defined in claim 17 and wherein R₆ is a linear or branched alkyl group having 10-20 carbon atoms.

20. The UV-absorbing polymer according to claim 17, wherein the synthetic amine rich polymer is represented by any of formulas (IV)-(VI): 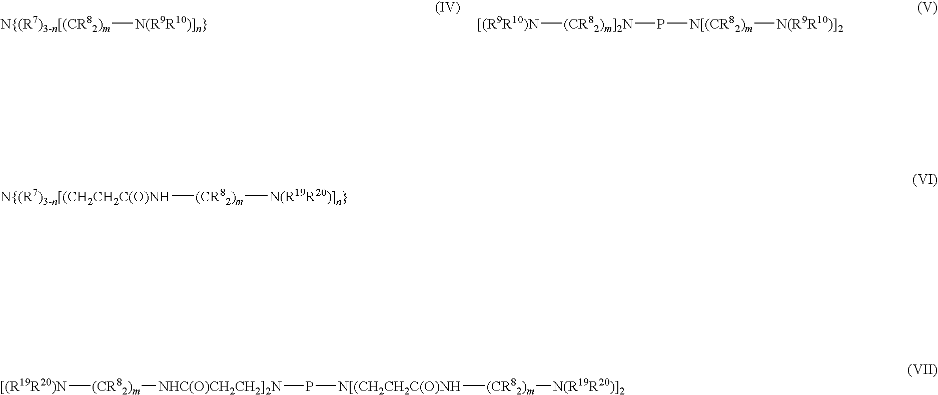
wherein R⁷ is selected from the group consisting of hydrogen atoms, linear or branched C₁-C₂₀ alkyl groups and —[(CR¹⁷₂)_{q}—X]ₒ—R¹⁸ groups, wherein X is O or NH;
m is 2, 3 or 4;
n is 2 or 3;
o is 1-10;
q is 2 or 3;
P is selected from the group consisting of —(CR⁸₂)ₘ—, C₆-C₁₂ arylene groups, C₆-C₁₂ cycloalkylene groups and —[(CR¹⁷₂)_{q}—X]ₚ—C(R¹⁷)₂]— groups, wherein X is O or NH and p is 1-10;
R⁸ is a hydrogen atom or a linear or branched C₁-C₆ alkyl group;
R⁹ and R¹⁰ are each independently selected from the group consisting of hydrogen atoms, linear or branched C₁-C₆ alkyl groups and groups of the formula —(CR¹⁷₂)_{q}NR¹¹R¹², provided that R⁹ and R¹⁰ are not both a linear or branched C₁-C₆ alkyl group;
R¹¹ and R¹² are each independently selected from the group consisting of hydrogen atoms, linear or branched C₁-C₆ alkyl groups and groups of the formula —(CR¹⁷₂)_{q}NR¹³R¹⁴, provided that R¹¹ and R¹² are not both a linear or branched C₁-C₆ alkyl group;
R¹³ and R¹⁴ are each independently selected from the group consisting of hydrogen atoms, linear or branched C₁-C₆ alkyl groups and groups of the formula —(CR¹⁷₂)_{q}NR¹⁵R¹⁶, provided that R¹³ and R¹⁴ are not both a linear or branched C₁-C₆ alkyl group;
R¹⁵ and R¹⁶ are each independently selected from the group consisting of hydrogen atoms and linear or branched C₁-C₆ alkyl groups, provided that R¹⁵ and R¹⁶ are not both a linear or branched C₁-C₆ alkyl group;
R¹⁷ is a hydrogen atom or a methyl group, provided that at least one R¹⁷ is a hydrogen atom;
R¹⁸ is selected from the group consisting of hydrogen atoms, linear or branched C₁-C₂₀ alkyl groups and —[(CR¹⁷₂)_{q}—X]ₒ—R¹⁸ groups as defined above;
R¹⁹ and R²⁰ are each independently selected from the group consisting of hydrogen atoms, linear or branched C₁-C₆ alkyl groups and groups of the formula —CH₂CH₂C(O)NH—(CR⁸₂)ₘ—N(R²¹R²²), provided that R¹⁹ and R²⁰ are not both a linear or branched C₁-C₆ alkyl group;
R²¹ and R²² are each independently selected from the group consisting of hydrogen atoms, linear or branched C₁-C₆ alkyl groups and groups of the formula —CH₂CH₂C(O)NH—(CR⁸₂)ₘ—N(R²³R²⁴), provided that R²¹ and R²² are not both a linear or branched C₁-C₆ alkyl group;
R²³ and R²⁴ are each independently selected from the group consisting of hydrogen atoms, linear or branched C₁-C₆ alkyl groups and groups of the formula —CH₂CH₂C(O)NH—(CR⁸₂)ₘ—N(R²⁵R²⁶), provided that R²³ and R²⁴ are not both a linear or branched C₁-C₆ alkyl group; and
R²⁵ and R²⁶ are each independently selected from the group consisting of hydrogen atoms and linear or branched C₁-C₆ alkyl groups, provided that R²⁵ and R²⁶ are not both a linear or branched C₁-C₆ alkyl group.

21. The UV-absorbing polymer according to claim 20, wherein the synthetic amine rich polymer has the formula (IV) or (V).

22. The UV-absorbing polymer according to claim 17, wherein polymer has a UV-absorption of at least 20 a.u./g.L at 375 nm.

23. The UV-absorbing polymer according to claim 17, wherein less than 10% of the total absorption between 250 and 600 nm of the UV-absorbing polymer is above 400 nm.

24. The UV-absorbing polymer according to claim 17, wherein at least 75% of the total absorption between 250 and 600 nm of the UV-absorbing polymer is between 315 and 400 nm.

25. The UV-absorbing polymer according to claim 17, wherein the amine rich synthetic polymer is a dendrimer.

26. The UV-absorbing polymer according to claim 17, wherein the UV-absorbing polymer has a number average molecular weight Mₙ of 1,000 to 10,000 g/mol.

27. A process for the preparation of a UV-absorbing polymer comprising reacting an aminobutadiene according to general formula (I): 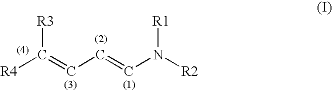
wherein R₁ and R₂, which may be the same or different, are each independently selected from the group consisting of hydrogen atoms, alkyl groups having 1-20 carbon atoms, acyl groups having 1-10 carbon atoms and aryl groups having 6-20 carbon atoms, provided that R₁ and R₂ do not simultaneously represent hydrogen atoms, optionally R₁ and R₂ can combine and form a cyclic amino group or R₁ or R₂ can combine with C(1) and from a cyclic amino group;
each of R₁ and R₂ are optionally substituted by one or more carboxylic acid moieties; R₃ is selected from the group consisting of —COOH, —COOR₅, —COR₅, —CN and SO₂R₅, and R₄ is selected from the group consisting of —COOH, —COOR₆, —COR₆, —CN and —SO₂R₆, wherein R₅ and R₆, which may be the same or different, are each independently selected from the group consisting of alkyl groups having 1-25 carbon atoms, alkyl groups having 1-25 carbon atoms and one or more carbon-carbon double bonds, wherein the alkyl groups optionally comprise one or more hetero-atoms selected from the group consisting of O, N, Si and S, and aryl groups having 6-20 atoms; optionally R₅ and R₆ can combine and form a ring structure which is optionally substituted and which optionally comprises N, O and/or carbonyl groups;
the UV absorbing polymer having a number average molecular weight Mₙ of 1,000 g/mol to 100,000 g/mol; and having a UV-absorption of at least 5.6 a.u./g.L at 375 nm; with a synthetic amine rich polymer.

28. The process according to claim 27, wherein the aminobutadiene is represented by formula (IX): 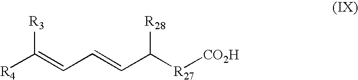
wherein R₂₇ and R₂₈, which may be the same or different, each represent an alkyl group having 1-10 carbon atoms; R₈ can also represent a hydrogen atom; optionally R₂₇ and R₂₈ can combine and form a cyclic amino group.

29. The process according to claim 28, wherein the aminobutadiene represented by formula (IX) is synthesized by:
(a) reacting the aminobutadiene according to formula (VII) with a carboxylic acid derivative according to formula (VIII) to form an aminobutadiene derivative according to formula (IX): 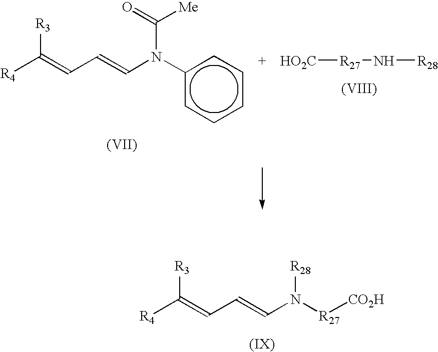
(b) wherein R₂₇ and R₂₈, which may be the same or different, each represent an alkyl group having 1-10 carbon atoms; R₈ can also represent a hydrogen atom; optionally R₂₇ and R₂₈ can combine and form a cyclic amino group.

30. The process according to claim 27, wherein the synthetic amino rich polymer is represented by any one of formulas (IV)-(VI): 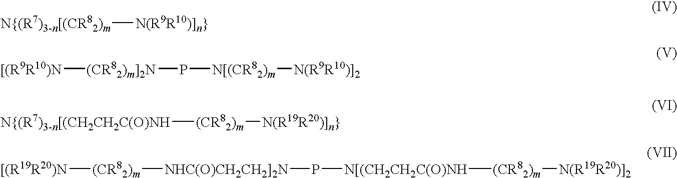
wherein R⁷ is selected from the group consisting of hydrogen atoms, linear or branched C₁-C₂₀ alkyl groups and —[(CR¹⁷₂)_{q}—X]ₒ—R¹⁸ groups, wherein X is O or NH;
m is 2, 3 or 4;
n is 2 or 3;
o is 1-10;
q is 2 or 3;
P is selected from the group consisting of —(CR⁸₂)ₘ—, C₆-C₁₂ arylene groups, C₆-C₁₂ cycloalkylene groups and —[(CR¹⁷₂)_{q}—X]ₚ—C(R¹⁷)₂]— groups, wherein X is O or NH and p is 1-10;
R⁸ is a hydrogen atom or a linear or branched C₁-C₆ alkyl group;
R⁹ and R¹⁰ are each independently selected from the group consisting of hydrogen atoms, linear or branched C₁-C₆ alkyl groups and groups of the formula —(CR¹⁷₂)_{q}NR¹¹R¹², provided that R⁹ and R¹⁰ are not both a linear or branched C₁-C₆ alkyl group;
R¹¹ and R¹² are each independently selected from the group consisting of hydrogen atoms, linear or branched C₁-C₆ alkyl groups and groups of the formula —(CR¹⁷₂)_{q}NR¹³R¹⁴, provided that R¹¹ and R¹² are not both a linear or branched C₁-C₆ alkyl group;
R¹³ and R¹⁴ are each independently selected from the group consisting of hydrogen atoms, linear or branched C₁-C₆ alkyl groups and groups of the formula —(CR¹⁷₂)_{q}NR¹⁵R¹⁶, provided that R¹³ and R¹⁴ are not both a linear or branched C₁-C₆ alkyl group;
R¹⁵ and R¹⁶ are each independently selected from the group consisting of hydrogen atoms and linear or branched C₁-C₆ alkyl groups, provided that R¹⁵ and R¹⁶ are not both a linear or branched C₁-C₆ alkyl group;
R¹⁷ is a hydrogen atom or a methyl group, provided that at least one R¹⁷ is a hydrogen atom;
R¹⁸ is selected from the group consisting of hydrogen atoms, linear or branched C₁-C₂₀ alkyl groups and —[(CR¹⁷₂)_{q}—X]ₒ—R¹⁸ groups as defined above;
R¹⁹ and R²⁰ are each independently selected from the group consisting of hydrogen atoms, linear or branched C₁-C₆ alkyl groups and groups of the formula —CH₂CH₂C(O)NH—(CR⁸₂)ₘ—N(R²¹R²²), provided that R¹⁹ and R²⁰ are not both a linear or branched C₁-C₆ alkyl group;
R²¹ and R²² are each independently selected from the group consisting of hydrogen atoms, linear or branched C₁-C₆ alkyl groups and groups of the formula —CH₂CH₂C(O)NH—(CR⁸₂)ₘ—N(R²³R²⁴), provided that R²¹ and R²² are not both a linear or branched C₁-C₆ alkyl group;
R²³ and R²⁴ are each independently selected from the group consisting of hydrogen atoms, linear or branched C₁-C₆ alkyl groups and groups of the formula —CH₂CH₂C(O)NH—(CR⁸₂)ₘ—N(R²⁵R²⁶), provided that R²³ and R²⁴ are not both a linear or branched C₁-C₆ alkyl group;
R²⁵ and R²⁶ are each independently selected from the group consisting of hydrogen atoms and linear or branched C₁-C₆ alkyl groups, provided that R²⁵ and R²⁶ are not both a linear or branched C₁-C₆ alkyl group; and
said synthetic amino rich polymer having —NHR* end groups, to form a UV-absorbing polymer according to formula (X), wherein R* is hydrogen or a linear or branched C₁-C₆ alkyl group:

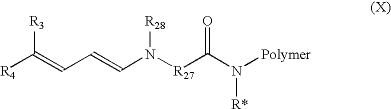

31. A composition for protection against UV radiation comprising a UV absorbing polymer according to claim 17.

32. The composition according to claim 31 comprising less than 18 wt % of the UV absorbing polymer.
